SEQUENCE LISTING (3 sequences)

SEQ ID NO: 1 - protein, 1273 aa, SARS coronavirus
  YP_009724390.1 surface glycoprotein of the Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 26 aa, Artificial Sequence
  Domain of the S protein responsible of the interaction with ACE2 receptor
EGFNCYFPLQSYGFQPTNGVGYQPYR

SEQ ID NO: 3 - protein, 26 aa, Artificial Sequence
  Mimocov2 peptide according to the invention
EGFNSYFPLQSYGCQPTNGVGYQCYR

(19) United States
(12) Patent Application Publication
(10) Pub. No.: US 2024/0233860 A1
(43) Pub. Date: Jul. 11, 2024

(54) METHOD FOR MODELING CYCLIC 3D EPITOPES TO BE USED IN THE DEVELOPMENT OF VACCINES

(71) Applicant: SYNTHETIC VACCINES LTD, Manchester Essex (GB)

(72) Inventor: Patrick RAMBAUD, Mandelieu-la-Napoule (FR)

(73) Assignee: SYNTHETIC VACCINES LTD, Manchester Essex (GB)

(21) Appl. No.: 18/558,825

(22) PCT Filed: May 5, 2022
(86) PCT No.: PCT/EP2022/062156

§ 371 Date: Nov. 3, 2023

(30) Foreign Application Priority Data
May 5, 2021 (EP) 21172344.0

(51) Int. Cl. G16B 15/20; A61K 39/00; C07K 14/005

(52) U.S. Cl. CPC G16B 15/20; C07K 14/005; A61K 39/00; C12N 2770/20022; C12N 2770/20034

(57) ABSTRACT

A method for modeling a cyclic 3D epitope from a surface of a target antigen including the steps of: a) identifying, in a 3D model representation of the target antigen, one or more conserved region(s) susceptible to be/constitute a 3D epitope, the one or more conserved region(s) possessing one or more charged amino acid(s) selected from the group of arginine (Arg), lysine (Lys), histidine (His), aspartic acid (Asp) and glutamic acid (Glu); b) performing molecular modeling on the one or more conserved region(s) identified in step a); c) providing a linear amino acid sequence susceptible to gather the one or more conserved region(s) in the 3D epitope; d) cyclizing in silico the linear amino acid sequence, so as to mimic the 3D epitope as it is in the target antigen; e) obtaining a modelized cyclic 3D epitope.

### FIELD

The present invention relates to the development of vaccines, in particular synthetic vaccines, by the mean of modeling cyclic 3D epitopes.

### BACKGROUND

The interest of synthetic peptide immunogens as an approach to generate specific immunological reagents, such as antibodies, and in particular neutralizing antibodies, has increased noticeably in the past decades.

In theory, many peptide sequences can be in immunogenic, but in practice, not all are equally effective at eliciting antibodies that cross-react with the intact cognate protein. It is now acknowledged that many factors may influence the success of using peptide immunogens to raise specific antibodies. These include elements such as the number of peptides from one protein sequence to be used, the availability and accuracy of sequence data, the predicted secondary and tertiary structures of the intact protein and finally, the ease of synthesis of specific sequences. Even though continual improvements to synthesis methodologies means that the latter aspect is less significant than in the past, certain sequences can still be problematic (Hancock and O'Reilly; 2005. Methods in Molecular Biology, vol. 295: Immunochemical Protocols, Third Edition. Chapter 2. Edited by: R. Burns @ Humana Press Inc., Totowa, NJ).

In recent years, scientists have developed a mimotope approach. A mimotope is a peptide able to mimic an epitope. Epitopes are specific surfaces of antigenic proteins and are recognized by antibodies. Epitopes can be linear or tridimensional (3D), but 3D-epitopes are recognized by 80% of immunoglobulins (IgG) and are much more specific. The main approach to design 3D-epitopes is to use molecular modeling to design a peptide with a 3D structure able to mimic an epitope.

So far, two main strategies have been developed in the field. The first approach is the in vivo production of a peptide interest by the means of the technology relying upon the expression of recombinant cDNA into a heterologous living organism, in particular bacteria. The second approach is the chemically-based in vitro synthesis of peptides.

Once synthetic peptides have been produced, vaccination with synthetic peptides obtained from Fmoc chemistry have often no stable 3D structure and are useful only to recognize linear epitope.

There is therefore a need to overcome these drawbacks and to provide the state of the art with reproducible methods for modeling stable and robust 3D epitopes, which may be used for the development of synthetic vaccines.

### SUMMARY

A first aspect of the invention relates to a method for modeling a cyclic 3D epitope from a surface of a target antigen comprising the steps of:
a) identifying, in a 3D model representation of the target antigen, one or more conserved region(s) susceptible to be/constitute a 3D epitope, the one or more conserved region(s) possessing one or more charged amino acid(s) selected in the group consisting of arginine (Arg), lysine (Lys), histidine (His), aspartic acid (Asp) and glutamic acid (Glu);
b) performing molecular modeling on the one or more conserved region(s) identified in step a);
c) providing a linear amino acid sequence susceptible to gather the one or more conserved region(s) in the 3D epitope;
d) cyclizing in silico the linear amino acid sequence, so as to mimic the 3D epitope as it is in the target antigen;
e) obtaining a modelized cyclic 3D epitope.

In some embodiments, the cyclic 3D epitope is a mimotope. In certain embodiments, the cyclic 3D epitope is synthetic. In some embodiments, the target antigen is selected in the group comprising or consisting of a bacterial antigen, a viral antigen and a cancer antigen. In certain embodiments, step a) is performed by aligning homologous and/or variant sequences of the one or more conserved region(s) to obtain atomic coordinates. In some embodiments, the one or more conserved region(s) is/are at the surface of the 3D model representation of the target antigen. In certain embodiments, step b) comprises performing one or more cycle(s) of energy minimization of a force field induced by atomic coordinates, in particular by the means of one or more energy minimization algorithm(s). In some embodiments, the energy minimization algorithm(s) is/are associated to molecular dynamic steps. In certain embodiments, step d) is performed chemically, in particular by site specific crosslinking. In some embodiments, step d) is performed by substituting 2 amino acid residues by cysteine residues in the linear sequence obtained at step c), and wherein the dihedral angles between each of the alpha-carbons of each of the cysteine residues and any one of the adjacent amino acid residues are compatible with the formation of a disulfide bridge.

A further aspect of the invention relates to a cyclic 3D epitope obtained by the synthesis of a cyclic 3D epitope modelized by a method according to the instant invention.

In one aspect, the invention relates to a pharmaceutical or vaccine composition comprising the cyclic 3D epitope according to the instant invention.

The invention also relates to the cyclic 3D epitope or pharmaceutical composition according to the instant invention, for use in a method of vaccination of an individual.

In one aspect, the invention relates to the cyclic 3D epitope or pharmaceutical composition according to the instant invention, for use in the production of antibodies, in particular neutralizing antibodies.

Another aspect of the invention relates to the cyclic 3D epitope or pharmaceutical composition according to the instant invention, for use for eliciting an immunologic reaction in an individual.

### BRIEF DESCRIPTION OF THE DRAWINGS

FIG. 1 is a schematic representation of the SARS-COV-2 viral particle showing the SARS-COV-2 virus and the S (Spike) protein.

FIG. 2 is a scheme showing the epitope sequence that corresponds to only 5% of the S (Spike) protein, and which is part of the receptor binding domain (RBD) that is highly conserved in all SARS-COV-2 strains. “*” indicates the position of an identical amino acid residue. “:” indicates the position of amino acid residues that share similar properties (polarity or charge).

FIG. 3 is a scheme showing the structures of the Mimocov2 peptide (SEQ ID NO: 3). The primary structure corresponds to a sequence highly conserved in SARS-CoV-1 and SARS-COV-2 RBD that interacts directly with the ACE2 receptor. “*” indicates amino acid substitutions that have been introduced to make possible a disulfide bridge to stabilize the loop structure. A type 1 beta turn at the N-terminus and a loop at the C-terminus are the main secondary structures. The 3D structure was determined with the atomic coordinates of SARS-COV-1, the disulfide bridge was created only after energy minimization.

### DETAILED DESCRIPTION

In the present invention, the following terms have the following meanings:

“About” preceding a figure encompasses plus or minus 10%, or less, of the value of said figure. It is to be understood that the value to which the term “about” refers to is itself also specifically, and preferably, disclosed.

“Adjuvant” refers to a compound or a combination of compounds that potentiate(s) an immune response within a vaccine composition. In one embodiment, the adjuvant is used with a vaccine composition and thus potentiates the immune response towards an infection disease. For example, an adjuvant may increase the number of lymphocytes; increase the activation of lymphocytes; increase the fitness of lymphocytes; and/or increase the survival of lymphocytes.

“Comprise” is intended to mean “contain”, “encompass” and “include”. In some embodiments, the term “comprise” also encompasses the term “consist of”.

“Crosslinker” refers to a stretch of chemical or amino acid sequence that physically bind two domains or moieties of a peptide or polypeptide by the means of two reactive groups. In some embodiments, the crosslinker may be a homobifunctional (two identical reactive groups) or heterobifunctional (two distinct reactive group) chemical compound.

“Cyclic 3D epitope” refers to an epitope, being in the form of a peptide, in which the lateral chains of 2 non-contiguous amino acid residues are covalently linked, resulting in the peptide having a 3D structure. In one embodiment, the covalent link is chemically obtained, in particular by the means of a crosslinker. In one embodiment, the covalent link is a disulfide bridge in between 2 —SH groups belonging to 2 non-contiguous cysteine residues.

“Molecular Modeling” refers to an ensemble of techniques known to the skilled in the art and providing realistic 3D structures of a protein or peptide having a potential energy depending on Force Field. The potential energies of the force field can be modified with energy minimization and/or dynamic.

“Immunogenic composition” refers to a composition that is capable of eliciting an immune response in an individual upon contact with said individual.

“Immunogenic peptide” refers to a peptide capable of eliciting an immune response in an individual upon contact with said individual.

“Isolated peptide” refers to a peptide that is removed from the environment in which it has been synthesized. “Model” or “modeling” refers to the action of producing in silico a representation or simulation of a target antigen of interest. In some embodiment, the target of interest is a surface of a target antigen.

“Linker” refers to a stretch of chemical or amino acid sequence that physically separate two domains or moieties of a peptide or polypeptide. As used herein, “the peptide and the carrier protein are conjugated by the mean of a linker” is intended to mean that the peptide and the carrier protein are indirectly covalently associated through the linker.

“Mimotope peptide” refers to a peptide, in particular a synthetic peptide, that mimics epitopes of an antigen found in nature. As used herein, the mimotope may be represented by a linear peptide, and preferably a peptide with a 3D structure.

“Peptide” or “polypeptide” refers to a linear polymer of amino acids linked together by peptide bonds.

“Synthetic peptide” refers to a peptide that does not exist in nature and/or is synthesized chemically or by the mean of recombinant technologies.

“Vaccine composition” refers to a composition comprising one or more antigens and/or epitopes suitable for triggering an immune response against these antigens and/or epitopes in an individual that is the recipient of said composition.

With the goal of providing an easy, robust and reproducible method to design stable 3D epitopes, the inventors have shown that it was possible to block a 3D structure of a peptide, which mimics the surface epitope of an immunogenic target, in a conformation similar to a 3D epitope, in particular with a disulfide bridge. The method was named the “mimotope approach”.

In the mimotope approach, only one disulfide bridge is created. If there is a free cysteine or a natural disulfide bridge with a cysteine outside of the epitope, mutations have to be introduced with another amino acid residue, such as serine, for instance, to replace a free cysteine. It is advantageous to have only one disulfide bridge because two or three disulfide bridges could produce different conformations for a same sequence.

In order to determine the optimal localization for the disulfide bridge, the method of the invention uses a specific force field equation called MImotope MOdeling Force Field (MIMOFF) and, optionally, a minimization algorithm mixed with dynamics steps. This mathematical approach uses a force field corresponding to a set of equations providing a potential energy to a peptide chain regarding its atomic 3D coordinates. The purpose of energy minimization is to reduce the Force Field energy with energy minimization algorithms to an optimal potential energy with a negative value regarding the van der Waals energy.

This invention relates to a method for modeling a cyclic 3D epitope from a surface of a target antigen comprising the steps of:
a) identifying, in a 3D model representation of the target antigen, one or more conserved region(s) susceptible to be/constitute a 3D epitope, the one or more conserved region(s) possessing one or more charged amino acid(s) selected in the group consisting of arginine (Arg), lysine (Lys), histidine (His), aspartic acid (Asp) and glutamic acid (Glu);
b) performing molecular modeling on the one or more conserved region(s) identified in step a);
c) providing a linear amino acid sequence susceptible to gather the one or more conserved region(s) in the 3D epitope;
d) cyclizing in silico the linear amino acid sequence, so as to mimic the 3D epitope as it is in the target antigen;
e) obtaining a modelized cyclic 3D epitope.

In some embodiments, step b) is performed using the following equation E to calculate the energy of the force field:

$$E = \frac{1}{2} {\sum }_{f} {{K}_{f} ( 1 - {e}^{- α ( r - {r}_{0} )} )}^{2} + \frac{1}{2} {\sum }_{θ} {{H}_{θ} ( θ - {θ}_{0} )}^{2} + {\sum }_{Φ} {H}_{Φ} ( 1 + cos {n}_{Φ} ) + {\sum }_{r} \frac{A}{{r}^{12}} - \frac{B}{{r}^{6}} + {\sum }_{r} \frac{q 1 q 2}{D r}$$

wherein E is the energy of the force field, r is the radius between two covalently bound atoms, r₀ is the van der Waals ideal radius at 298 K, θ is the valence angle between two covalent bonds, ϕ is the dihedral angle for SP3 carbons, n is the multiplicity or periodicity of the dihedral angle, the parameters Hϕ, and Hθ are the respective force constants and the variables with the subscript 0 are the respective equilibrium values, A is the distance between two atoms, B is the double distance between two non-bound atoms, q is the atomic charge and D is Debie constant.

The above equation E is thereby referred to as MImotope MOdeling Force Field (MIMOFF).

It is to be understood that the equation E used in step b) can be split into 5 distinct equations as follow:

$$E 1 = \frac{1}{2} {\sum }_{f} {{K}_{f} ( 1 - {e}^{- α ( r - {r}_{0} )} )}^{2} \qquad Equation E1$$

$$E2 = \frac{1}{2} {\sum }_{θ} {{H}_{θ} ( θ - {θ}_{0} )}^{2} \qquad Equation E2$$

$$E3 = {\sum }_{Φ} {H}_{Φ} ( 1 + cos {n}_{Φ} ) \qquad Equation E3$$

$$E4 = {\sum }_{r} \frac{A}{{r}^{12}} - \frac{B}{{r}^{6}} \qquad Equation E4$$

$$E5 = {\sum }_{r} \frac{q 1 q 2}{D r} \qquad Equation E5$$

wherein equation E1 is an exponential equation reflecting the energy associated with the radius between two covalently bound atoms; equation E2 is harmonic equation that maintains the energy due to the geometry of electron orbitals involved in covalent bonding, reflecting the energy associated with the angles between particles (the angles between three covalently bounded atoms being respectively 106.7° and 120° regarding carbon hybridization (SP3 and SP2); equation E3 is a sinusoidal equation representing the energy for twisting a bond due to bond order and neighboring bonds or lone pairs of electrons, equation E4 reflects the van der Waals energy for non-bound atoms, and equation E5 reflects the atomic charge.

In some embodiments, equation E3 is adapted to the orbital hybridization of the carbon atom. In some embodiments, equation E3 reflects the energy associated with dihedral angle for SP2 carbon atoms. In some embodiments, E3 reflects the energy associated with dihedral angle for SP3 carbon atoms.

In some embodiments, equations E2, E3 and E5 are derived from AMBER force field. In some embodiments, equation E4 is derived from CHARMM force field. Both force fields are known to the skilled in the art.

In some embodiments, equation E1 is specific to MIMOFF, where a non-harmonic function is used to describe the van der Waals energy contrary to AMBER or CHARMM force fields that use a harmonic function. In some embodiments, equation E1 provides negative energy when two bound atoms respect the optimal van der Waals radius.

In some embodiments, the cyclic 3D epitope is a mimotope.

In certain embodiments, the cyclic 3D epitope is synthetic.

In some embodiments, the epitope is a peptide. In practice, the peptide of the invention may be synthesized by any suitable method known from the state of the art, or a method derived therefrom. Illustratively, methods for synthesizing a peptide according to the invention may be disclosed, e.g., by Lloyd-Williams et al. (1997; Chemical approaches to the synthesis of peptides and proteins. Boca Raton: CRC Press. 278), Merrifield (1963; Journal of the American Chemical Society. 85, 2149-54), Lewandowski et al. (2013; Science, Vol. 339(6116), 189-193).

In some embodiments, the target antigen is selected in the group comprising or consisting of a bacterial antigen, a viral antigen and a cancer antigen.

As used herein, “bacterial antigen” and “viral antigen” refer to antigen of bacterial or viral origin, respectively, which may elicit an immune response in an individual, in particular a vertebrate individual, more particularly a non-human mammalian animal or a human.

In certain embodiments, the bacterial antigen and/or viral antigen may be associated with an infectious disease, in particular an infectious disease selected in a group consisting of acute flaccid myelitis (AFM), anaplasmosis, anthrax, babesiosis, botulism, brucellosis, campylobacteriosis, carbapenem-resistant infection (CRE/CRPA), chancroid, Chikungunya virus infection (Chikungunya), chlamydia, Ciguatera (Harmful Algae Blooms (HABs)), Clostridium Difficile infection, Clostridium Perfringens (Epsilon Toxin), Coccidioidomycosis fungal infection (Valley fever), COVID-19 (Coronavirus Disease 2019), Creutzfeldt-Jacob disease, transmissible spongiform encephalopathy (CJD), Cryptosporidiosis (Crypto), cyclosporiasis, Dengue (Dengue Fever), diphtheria, Shiga toxin-producing E. coli infection (STEC), Eastern Equine Encephalitis (EEE), Ebola Hemorrhagic Fever (Ebola), Ehrlichiosis, arboviral or parainfectious encephalitis, non-polio Enterovirus Infection (Non-Polio Enterovirus), D68 Enterovirus Infection (EV-D68), Giardiasis (Giardia), Glanders, Gonococcal Infection (Gonorrhea), Granuloma inguinale, Type B Haemophilus Influenza disease (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection (Histoplasmosis), Human Immunodeficiency Virus/AIDS (HIV/AIDS), Human Papillomavirus (HPV), Influenza (Flu), lead poisoning, Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Viral Meningitis, Bacterial Meningococcal Disease, Middle East Respiratory Syndrome Coronavirus (MERS-COV), Multisystem Inflammatory Syndrome in Children (MIS-C), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), bubonic, septicemic, pneumonic plague (plague), Pneumococcal Disease (Pneumonia), Poliomyelitis (Polio), Powassan, Psittacosis (Parrot Fever), Pthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella (German Measles), Salmonellosis gastroenteritis (Salmonella), Scabies Infestation (Scabies), Scombroid, Septic Shock (Sepsis), Severe Acute Respiratory Syndrome (SARS), Shigellosis gastroenteritis (Shigella), Smallpox, Methicillin-resistant Staphylococcal Infection (MRSA), Staphylococcal Food (Enterotoxin-B) poisoning (Staph Food Poisoning), vancomycin intermediate Staphylococcal infection (VISA), vancomycin resistant Staphylococcal infection (VRSA), Group A (invasive) Streptococcal Disease (Strep A (invasive)), Group B Streptococcal Disease (Strep-B), Streptococcal Toxic-Shock Syndrome (STSS, TSS), Syphilis, Tetanus infection (tetani, Lock Jaw), Trichomoniasis (Trichomonas infection), Trichonosis Infection (Trichinosis), Tuberculosis (TB), Tularemia (Rabbit fever), Group D Typhoid Fever, Typhus, bacterial Vaginosis (Yeast Infection), Vaping-Associated Lung Injury (e-Cigarette Associated Lung Injury), varicella (chickenpox), Vibrio cholerae infection (Cholera), vibriosis (Vibrio), Viral Hemorrhagic Fever (Ebola, Lassa, Marburg), West Nile Virus, Yellow Fever, Yersinia infection, and Zika virus infection (Zika).

As used herein, the term “cancer antigen” is intended to refer to an antigen that may be found in high amounts in the blood of patients with certain types of cancer.

Non-limitative examples of cancers include acute lymphoblastic leukemia, acute myeloblastic leukemia adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, ovarian cancer, pancreatic cancer, prostate cancer, salivary gland cancer, sarcoma, squamous cell carcinoma, testicular cancer, thyroid cancer, urothelial carcinoma, and uveal melanoma.

In practice, said cancer is a blood cancer or a solid cancer.

As used herein, the term “blood cancer”, also referred to as “hematologic cancer”, encompasses any cancer involving uncontrolled proliferation of blood cells, in particular white blood cells. Blood cancers includes immunoblastic lymphadenopathy, leukemia, lymphoma (Hodgkin and non-Hodgkin lymphomas) and myeloma.

As used herein, the term “solid cancer” encompasses any cancer (also referred to as malignancy) that forms a discrete tumor mass, as opposed to cancers (or malignancies) that diffusely infiltrate a tissue without forming a mass. Solid cancer include melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, glioblastoma, prostate carcinoma, ovarian cancer and pancreatic carcinoma.

Non-limitative examples of cancer antigen include alpha-fetoprotein (AFP), cancer antigen 125 (CA125), cancer antigen 15-3 (CA15-3), carbohydrate antigen 19-9 (CA19-9), carcinoembryonic antigen (CEA), human chorionic gonadotropin (hCG or beta-hCG), and prostate-specific antigen (PSA).

In some embodiments, the cancer antigen is a neoantigen.

As used herein, the term “neoantigen” is a newly formed antigen that has not been previously recognized by the immune system. Neoantigens and, by extension, neoantigenic determinants (or neoepitopes), can be formed when a protein undergoes further modification within a biochemical pathway such as glycosylation, phosphorylation or proteolysis.

Neoantigen mutations may be identified by comparing DNA isolated from tumor versus normal sources.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the invention.

In certain embodiments, the target antigen is a polypeptide or a protein.

In certain embodiments, the 3D model representation of the target antigen may be available from the databases. In some embodiments, the 3D model representation of the target antigen may be obtained by the means of crystallography and/or NMR (nuclear magnetic resonance). In certain embodiments, the 3D model representation of the target antigen may be obtained upon modeling from 3D model representation(s) of antigen(s) that is/are homologous to the target antigen. In some embodiments, the 3D model representation of the target antigen may be obtained by the means of molecular modeling to predict 3D structures.

Non-limitative examples of algorithms to minimize energy of a force field induced from atomic coordinates include Steepest Descent, Conjugate Gradients, and the like.

In certain embodiments, step a) is performed by aligning homologous and/or variant sequences of the one or more conserved region(s) to obtain atomic coordinates.

In practice, step a) may be performed by aligning sequences of homologous polypeptides or proteins. As used herein, the term “homologous polypeptides or proteins” is intended to refer to polypeptides or proteins that share a substantial amino acid sequences identity. In practice, homologous polypeptides or proteins encompass orthologous polypeptides or proteins, paralogous polypeptides or proteins and xenologous polypeptides or proteins.

As used herein, the term “sequences identity” when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “homology” from InsightII). Identity of related polypeptides or nucleic acid sequences can be readily calculated by known methods. Preferred methods for determining identity are designed to give the largest match between the tested sequences. Methods of determining identity are described in publicly available computer programs. Preferred computer program-based methods for determining identity between two sequences include the GCG program package, including GAP (Devereux et al., Nucl. Acid. Res. 2, 387 (1984); Genetics Computer Group, University of Wisconsin, Madison, Wis.), BLASTP, BLASTN, TBLASTN and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., see above). The well-known Smith Waterman algorithm may also be used to determine identity. In one embodiment, the “identity” refers to a parameter measured over the entire length of the sequence to which it refers.

In practice, amino acid sequences identity may be assessed by any suitable algorithm from the state of the art. Non-limiting examples of algorithm include CLUSTAL, CLUSTAL W, BLAST P. LALIGN, and the like.

Illustratively, the amino acid identity percentage may also be determined using the CLUSTAL W software (version 1.83) the parameters being set as follows:
for slow/accurate alignments: (1) Gap Open Penalty: 10.00; (2) Gap Extension Penalty: 0.1; (3) Protein weight matrix: BLOSUM;
for fast/approximate alignments: (4) Gap penalty: 3; (5) K-tuple (word) size: 1; (6) No. of top diagonals: 5; (7) Window size: 5; (8) Scoring Method: PERCENT.

In some embodiments, the one or more conserved region(s) is/are at the surface of the 3D model representation of the target antigen.

As used herein, the term “at the surface” is intended to mean the one or more conserved region(s) is/are exposed to the solvent when the target antigen is in an aqueous solution.

In certain embodiments, step b) comprises performing one or more cycle(s) of energy minimization of a force field induced by atomic coordinates, in particular by the means of one or more energy minimization algorithm(s). Energy minimization algorithm(s) are known in the art.

In some embodiments, the energy minimization is achieved by using the derivate of a force field equation. In some embodiments, the energy minimization is achieved by using the derivate of the equation used in step b) of the present invention, preferably of equation E (MIMOFF).

In some embodiments, the derivate of the force field equation provides the atomic coordinates with the lowest potential energy of the force field.

As used herein, the term “energy minimization” is intended to refer to the approach to find a set of coordinates representing the minimum energy conformation for a given structure of a peptide, polypeptide or protein. It is commonly acknowledged in the state of the art that the energy of a peptide, polypeptide or protein can be characterized as a function of its atomic coordinates. In practice, this energy function may rely in several parameters, such as, e.g., the bond energy and angle energy, representative of the covalent bonds and bond angles, respectively; the dihedral energy resulting from the dihedral angles; the van der Waals forces that determine the steric hindrance of atoms; and electrostatic forces, which correspond to the long-range forces between charged and partially charged atoms.

In practice, non-limitative examples of algorithms for energy minimization include Steepest Descent and Conjugate Gradient. In some embodiments, the energy minimization may be performed by using the module Discover of InsightII® software with the equation used in step b) of the present invention, preferably equation E (MIMOFF).

In some embodiments, the energy minimization algorithm(s) is/are associated to molecular dynamic steps.

As used herein, “molecular dynamic” is intended to refer to the approach to resolve the stop of energy minimizations in an energy wheel.

In practice, molecular dynamic is associate to Steepest Descent algorithm for energy minimization.

In some embodiments, the molecular dynamic steps comprise repeating one or more cycle(s) of the energy minimalization.

In some embodiments, the molecular dynamic steps are performed when the minimization algorithm is blocked.

In some embodiments, the molecular dynamic step comprises or consist of analyzing a trajectory of different potential energies of MIMOFF with energy minimization at different times. In some embodiments, the molecular dynamic steps comprise or consist of repeating one or more cycle(s) of the energy minimization using a different initial energy. In some embodiments, the molecular dynamic steps comprise or consist of repeating one or more cycle(s) of the energy minimalization using a different potential energy value.

According to the invention, step c) consists of providing a linear amino acid sequence susceptible to gather the one or more conserved region(s) in the 3D epitope. As used herein, the term “providing a linear amino acid sequence” is intended to mean “determining a linear amino acid sequence”. In one embodiment, the terms “providing” and “determining” are equivalent.

In some embodiments, two or more conserved regions in the 3D epitope may be arranged as a single linear amino acid sequence. In certain embodiments, the two or more conserved regions may be linked together by the mean of an amino acid linker, such as Glycine-rich amino acid linker, in particular comprising one or two Glycine repeats.

As used herein, the term “amino acid linker” refers to peptide which function is to separate in the space two or more peptide of interest, by providing a chain of peptide bonds. In practice, suitable amino acid linkers may be described in Chen et al. (Adv Drug Deliv Rev. 2013; 65(10): 1357-1369), Chichili et al. (Protein Sci. 2013; 22(2): 153-167), Crasto and Feng (Prot Engineer. 2000; 13(5), 309-312), Waldo et al. (Nat. Biotechnol. 1999; 17, 691-695).

In some embodiments, the amino acid linker has a length ranging from about 1 to about 100 amino acid residues, preferably from about 5 to about 50 amino acid residues, more preferably from about 10 to about 25 amino acid residues.

As used herein, the expression “from about 1 to about 100 amino acid residues” include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61,62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 amino acid residues.

In practice, the structure of the epitope of interest is maintained and stabilized by cyclization.

In certain embodiments, step d) is performed chemically, in particular by site specific crosslinking.

As used herein, the term “site specific crosslinking” is intended to refer to the process of chemically joining two or more selected amino acids by a covalent bond.

In some embodiments, site specific crosslinking may be performed by a crosslinker agent.

In practice, the specific crosslinking may be mediated by a crosslinker agent, including a homobifunctional crosslinker agent and a heterobifunctional crosslinker agent.

As used herein, “homobifunctional crosslinker agent” is intended to refer to a crosslinker agent that has identical reactive groups at either end of a spacer arm. Non-limitative examples of homobifunctional crosslinker agents include adipic acid dihydrazide (CAS Number 1071-93-8), 1,4-bis[3-(2-pyridyldithio) propionamido]butane (CAS Number 141647-62-3), disodium 4,4′-diisothiocyanatostilbene-2,2′-disulfonate (DIDS; CAS Number 3,3′-dithio-bis(propionimidate) dihydrochloride (DTBP; CAS Number 38285-78-8), dimethyl pimelinediimidate dihydrochloride (DMP; CAS Number 58537-94-3), 3,3′-dithiodipropionic acid di(N-hydroxysuccinimide ester) (DTSP; CAS Number 57757-57-0), ethylene glycol-bis(succinic acid N-hydroxysuccinimide ester) (CAS Number 70539-42-3), sebacic acid bis(N-succinimidyl) ester (DSSeb; CAS Number 23024-29-5), disuccinimidyl suberate (DSS; CAS Number 68528-80-3), and bis(sulfosuccinimidyl) suberate sodium salt (BS3; CAS Number 82436-77-9). In practice, homobifunctional crosslinker agents may crosslink identical free reactive groups within the lateral chain of amino acid residues. For example, free —OH groups may be localized on lateral chain of amino acid residues such as aspartic acid (Asp), glutamic acid (Glu), serine (Ser), threonine (Thr) and tyrosine (Tyr); free -NH2 groups may be localized on the lateral chain of amino acid residues such as asparagine (Asn), glutamine (Gln), arginine (Arg) and lysine (Lys).

As used herein, “heterobifunctional crosslinker agent” is intended to refer to a crosslinker agent that possesses different reactive groups at either end. Non-limitative examples of heterobifunctional crosslinker agents include acetylene-PEG4-maleimide, maleimide-PEG-succinimidyl ester, azido-PEG4-phenyloxadiazole methylsulfone, LC-SMCC (succinimidyl-4-(N-maleimidomethyl)cyclohexane-1-carboxy-(6-amidocaproate)), PDPH (3-(2-pyridyldithio)propionyl hydrazide) (CAS Number: 115616-51-8), SIAB (N-succinimidyl (4-iodoacetyl)aminobenzoate) (CAS Number: 72252-96-1), SMPH (succinimidyl-6-((b-maleimidopropionamido)hexanoate), Sulfo-SIAB (sulfo-succinimidyl (4-iodoacetyl)aminobenzoate), 3-(Maleimido)propionic acid N-hydroxy-succinimide ester (BMPS; CAS Number: 55750-62-4), 2-(2-((7-(tert-Butoxy)-7-oxoheptyl)oxy)ethoxy)acetic acid (OtBu-PEG2-acid), and N-(B-maleimidopropionic acid) hydrazide, trifluoroacetic acid salt (BMPH).

In practice, the crosslinker agent may be commercially available, e.g., from Merck®, Termofisher Scientific®, Sigma Aldrich®, and may be used following the manufacturer's instructions.

In some embodiments, step d) is performed by substituting 2 amino acid residues by cysteine residues in the linear sequence obtained at step c), and wherein the dihedral angles between each of the alpha-carbons of each of the cysteine residues and any one of the adjacent amino acid residues are compatible with the formation of a disulfide bridge.

As used herein, the term “disulfide bridge” refers to a covalent bond between two —SH groups from the lateral chains of free cysteines. To create a disulfide bridge, the —SH lateral chain of a first free cysteine has to be at less than 0.1 nm from another —SH lateral chain of a second free cysteine. The sp3 hybridity of the alpha carbon of each cysteine makes possible different orientation yet this flexibility is limited by the freedom degree of the dihedral angles of the peptide bonds with the neighbor amino acid residues. The size of the lateral chains of the neighbor amino acid residues may have a direct impact on the freedom of the dihedral angle.

In certain embodiment, step d) may be preceded a step of performing substitution(s) of one or more cysteine residue(s) by one or more amino acid residue(s) selected in the group consisting of alanine (Ala), serine (Ser), and threonine (Thr). preferably alanine (Ala) and serine (Ser), more preferably serine (Ser), in the linear sequence obtained at step c). In practice, the linear peptide at the end of step d) may comprise solely 2 cysteine residues, so as the formation of only 1 disulfide bridge may be allowed.

Another aspect of the invention also relates to a cyclic 3D epitope obtained by the synthesis of a cyclic 3D epitope modelized by a method according to the instant invention.

A further aspect of the invention pertains to a pharmaceutical or vaccine composition comprising the cyclic 3D epitope according to the instant invention.

In some embodiments, the pharmaceutical composition according to the invention comprises a pharmaceutically acceptable excipient. As used herein the term “pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, Food and Drugs Administration (FDA, in the United States) or the European Medicines Agency (EMA).

In some embodiments, the vaccine composition according to the invention comprises one or more adjuvant(s). As used herein, the term “adjuvant” refers to a component that potentiates the immune responses to an antigen and/or modulates it towards the desired immune responses. Thus, the incorporation of adjuvants into vaccine formulations is aimed at enhancing, accelerating and prolonging the specific immune response towards the desired response to vaccine antigens. Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised.

In some embodiments, the cyclic 3D epitope according to the invention may be conjugated to a carrier protein. Non-limiting examples of carrier proteins include Bovine serum albumin (BSA), Keyhole limpet hemocyanin (KLH), Multiple antigen peptides (MAPs), or Ovalbumin (OVA).

In one aspect, the invention relates to a cyclic 3D epitope or pharmaceutical composition according to the invention, for use in a method of vaccination of an individual.

One further aspect of the invention relates to a method for vaccinating an individual in need thereof, comprising the step of administering a therapeutically efficient amount of a cyclic 3D epitope or pharmaceutical composition according to the invention.

The invention also relates to a cyclic 3D epitope or pharmaceutical composition according to the instant invention, for use in the production of antibodies, in particular neutralizing antibodies.

In some aspect, the invention also relates to a method for the production of antibodies, in particular neutralizing antibodies, in an individual in need thereof, comprising the step of administering a therapeutically efficient amount of a cyclic 3D epitope or pharmaceutical composition according to the invention.

Another aspect of the invention pertains to a cyclic 3D epitope or pharmaceutical composition according to the instant invention, for use for eliciting an immunologic reaction in an individual.

A further aspect of the invention relates to a method for eliciting an immunologic reaction in an individual in need thereof, comprising the step of administering a therapeutically efficient amount of a cyclic 3D epitope or pharmaceutical composition according to the invention.

In some embodiments, the individual in need thereof is susceptible to be affected with an infectious disease or cancer.

### EXAMPLES

The present invention is further illustrated by the following examples.

### Example 1: Designing of a 3D Cyclic Epitope Against the S (spike) Protein of the SARS-COV-2

In late December 2019, several cases of pneumonia of unknown origin were reported from China, which in early January 2020 were announced to be caused by a novel coronavirus. The new emerging SARS-COV-2 virus (Severe Acute Respiratory Syndrome COronaVirus 2) is responsible of the ongoing outbreak of the coronavirus disease 2019 (COVID 19) (Walls et al., 2020). Even if an efficient drug treatment is found to cure COVID 19, only a vaccine will make possible to eradicate rapidly the spreading of this virus. A vaccine is the only alternative to confinement to limit mortality due to this virus.

Despite massive attempts to contain the disease in China, the virus has spread globally, and COVID-19 was declared a pandemic by the World Health Organization (WHO) in March 2020. COVID-19 induced worldwide more than 111 million cases and 2.46 million deaths in the last WHO report on COVID 19 (February 2020). A survey in Lombardy (Italia) on a cohort of volunteers (n=1591) critically ill and admitted in public hospitals from Feb. 20 to Mar. 18, 2020 showed that the global mortality was 26% with a majority of men. Mortality was 35% for volunteers >63 years old volunteers and 15% for <64 years old volunteer (Grasseli et al., 2020).

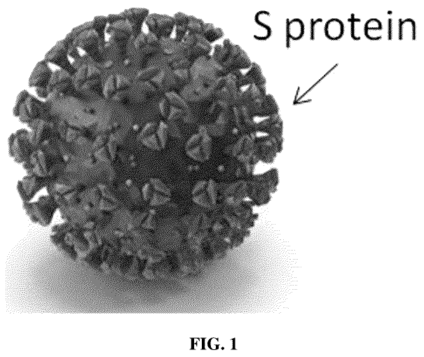

One of the main envelop proteins of the SARS-COV-2 virus is called the S (Spike) protein (FIG. 1). Volunteers infected can generate antibodies against nucleocapsid N protein and/or S protein but only antibodies triggering the receptor binding domain (RBD) at the top of S protein are neutralizing, which means that these antibodies help to cure from COVID-19.

To enter in human cells, the SARS-COV-2 S (Spike) protein interacts with a receptor called ACE2 for Angiotensin-Converting Enzyme 2 (Coutard et al., 2020 & Walls et al., 2020). A high affinity of the receptor binding domain (RBD) of the SARS-CoV-2 S (Spike) protein to the human ACE2 receptor, compared to other Corona virus could be the cause of the rapid viral spread of SARS-COV-2 in humans (Ortega et al., 2020). A preventive vaccine should trigger an IgG response against the RBD sequence to neutralize human cell infection.

A synthetic peptide obtained with Fmoc chemistry is the safest vaccinal approach and is currently used in more than 60 vaccines against SARS-COV-2. Synthetic vaccines are sterile and can be stable for three years after lyophilization. A short size peptide (less than 30 residues) is economically viable for mass vaccination. However, peptides have unstable structure (or Random Coil) and are useful only for linear epitopes.

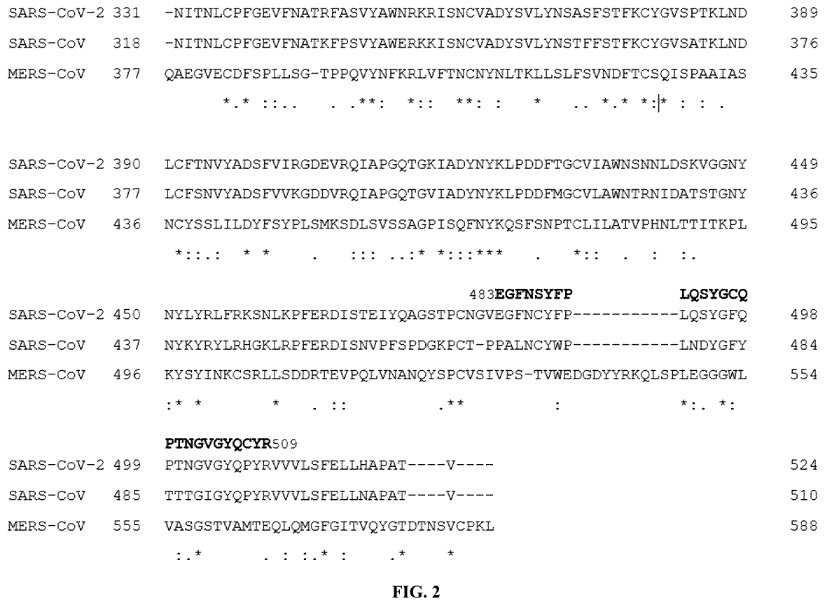

The first step for the design of the vaccine against the SARS-COV-2 (also referred to as Mimocov2 vaccine) was to identify structural motifs in the SARS-COV-2 RBD that bind to the ACE2 receptor (step a)). The 3D structure of ACE2 with SARS-COV-2 was not yet published when this design was made and the PDB file corresponding to the X ray structure of the ACE2 receptor with the S protein of SARS-COV-1 was used (Shang et al., 2020). Following sequences alignment analysis between the SARS-COV-1 sequence and the SARS-COV-2 sequence (SEQ ID NO: 1), it was possible to identify a highly conserved region in both SARS-COV-1 and SARS-COV-2 that interacts with the ACE2 receptor. Analysis of the Homology pulldown of InsightII® revealed a conserved region of the SARS-COV-2 RBD is shown in FIG. 2 that fits with a highly conserved region for SARS-COV-1 and all SARS-COV-2 variants from S (Spike) protein, from amino acid residue 483 to amino acid residue 509 (peptide of SEQ ID NO: 2; FIG. 2). This sequence displays low variability because it corresponds probably to an essential sequence for these two viruses to enter into human cells.

The sequence of the identified peptide has two structural motifs made of a beta turn (amino acid residue 483 to amino acid residue 491) and a beta turn at its two extremities (amino acid residue 506 to amino acid residue 509). The MERS-COV virus has an insertion in the middle of this sequence between these two structural motifs. This insertion probably decreases the affinity for the ACE2 receptor. The first loop that is strictly conserved in the three viruses is certainly a structural motif essential to fit into the ACE2 binding site.

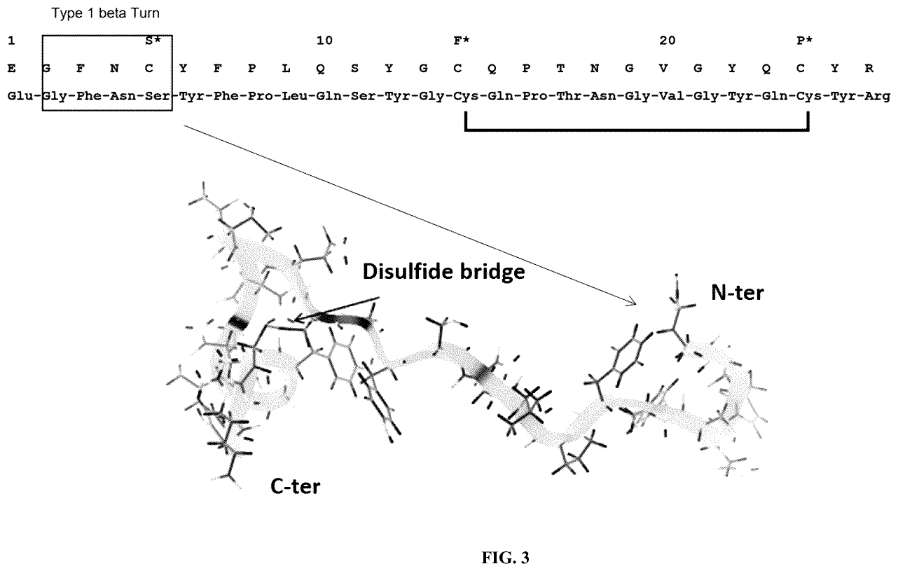

Molecular modeling was performed on the conserved region comprising the two structural motifs forming a loop, using the MIMOFF equation to calculate the force field (step b)). For this loop to conserve its 3D structure, the molecular modeling provides a model where the structure is rigidified by a disulfide bridge between a cysteine at position 497 and a cysteine at position 507. The molecular modeling taught that it was not possible to use the cysteine at position 488 to create a disulfide bridge with another part of the sequence. To fix the loop structure, the only possibility was thus to perform two amino acid substitutions, namely Phe497Cys and Pro507Cys as shown in FIG. 3. To avoid different possible disulfide bridges, a third substitution was introduced, namely Cys488Ser. The lateral chain of serine at position 488 is very close of cysteine with a —OH group instead of —SH and provides the same constraints for the dihedral angles. The resulting peptide was named Mimocov2 peptide (SEQ ID NO: 3).

The atomic coordinates of SARS-COV-1 (amino acid residue 483 to amino acid residue 509) were used as template to determine the atomic coordinates of the Mimocov2 peptide using Homology from the InsightII® software. From the X ray structure ACE2 receptor and the S protein of SARS-COV-1 (Shang et al., 2020), it was possible to model a peptide of less than 30 residues covering a loop of the SARS-COV-2 S (Spike) protein that interacts with the ACE2 receptor. This peptide belongs to the RBD. The peptide can overlap the backbone of the loop of the SARS-COV-2 S protein. The disulfide bridge that was introduced in the peptide blocks the structure as it is in the SARS-COV-2. Only one box containing amino acid residue 483 to amino acid residue 509 was used to determine the atomic coordinates. Using Discover (InsightII®), energy minimization cycles were then carried out with free cysteines to determine the potential structural changes due to the three substitutions. The Covalent Valence Force Field (CVFF) with the algorithm Steepest Descent® was used with free cysteines to not induce constraints. After three minimization cycles and few changes due to the three different side chains, the two —SH lateral chains of cysteines were still at the appropriate position to create a disulfide bridge.

The resulting peptide named Mimocov2 peptide (SEQ ID NO: 3) was thus confirmed to be the linear amino acid sequence able to gather the conserved region of the SARS-COV-2 RBD that bind to the ACE2 receptor (step c)). Its cyclization in silico leads to a modelized cyclic 3D epitope.

### Example 2: Synthesis of a Mimocov2 Peptide (Sequence SEQ ID NO: 3)

The Mimocov2 peptide synthesis was carried out with Fmoc/tBu chemistry using protecting group Trt for Cys (C), Asn (N) and Gln (Q), tBu for Glu, Ser (S), Thr (T) and Tyr (Y). For the chemical synthesis, Fmoc was deprotected with Piperidine/DMF (25/75).

Amino acid residues were coupled with HCTU and NMM with a ratio A/HCTU/NMM (5eq/5eq/10eq). Peptide deprotection was carried out with TFA/water/DTT (90/5/5).

The synthesized peptide was analyzed by HPLC on a Symmetry C18 Column, 100 Å, 5 μm, 4.6 mm×250 mm (from Waters®), following the manufacturers' instructions. The injection volume was 10 μL and the elution buffers A/B were:
A: 0.1% trifluoroacetic acid (TFA) in H₂O;
B: 0.1% TFA in acetonitril.

The results are depicted in Table 1 below.

TABLE 1

| Time (min) | RT | Height | Area | % Area |
|---|---|---|---|---|
| 1 | 4.570 | 3712 | 27904 | 0.20 |
| 2 | 4.900 | 1799 | 17592 | 0.13 |
| 3 | 5.367 | 4761 | 34332 | 0.24 |
| 4 | 5.407 | 5033 | 46622 | 0.33 |
| 5 | 6.051 | 1436 | 16882 | 0.12 |
| 6 | 6.396 | 2121 | 16875 | 0.12 |
| 7 | 11.011 | 3085 | 28742 | 0.20 |
| 8 | 11.419 | 1956 | 49198 | 0.35 |
| 9 | 11.731 | 4529 | 46984 | 0.33 |
| 10 | 11.933 | 4753 | 32463 | 0.23 |
| 11 | 12.071 | 9774 | 114064 | 0.81 |
| 12 | 12.350 | 9528 | 77806 | 0.55 |
| 13 | 12.533 | 18425 | 162218 | 1.15 |
| 14 | 12.640 | 22114 | 268428 | 1.91 |
| 15 | 12.917 | 60660 | 354055 | 2.52 |
| 16 | 13.062 | 882704 | 7734060 | 55.02 |
| 17 | 13.383 | 20736 | 130353 | 0.93 |
| 18 | 13.572 | 18235 | 192066 | 1.37 |
| 19 | 13.880 | 42494 | 469553 | 3.34 |
| 20 | 14.099 | 69636 | 634843 | 4.52 |
| 21 | 14.267 | 13059 | 102804 | 0.73 |
| 22 | 14.493 | 10199 | 134321 | 0.96 |
| 23 | 14.904 | 12501 | 254049 | 1.81 |
| 24 | 15.100 | 6454 | 55701 | 0.40 |
| 25 | 15.496 | 11607 | 180966 | 1.29 |
| 26 | 15.796 | 11165 | 163210 | 1.16 |
| 27 | 16.063 | 5644 | 72799 | 0.52 |
| 28 | 16.357 | 6195 | 68309 | 0.49 |
| 29 | 16.554 | 9076 | 113782 | 0.81 |
| 30 | 16.753 | 7329 | 82463 | 0.59 |
| 31 | 17.050 | 6548 | 64140 | 0.46 |
| 32 | 17.263 | 68776 | 660152 | 4.70 |
| 33 | 17.719 | 78413 | 806199 | 5.74 |
| 34 | 17.883 | 14723 | 139877 | 1.00 |
| 35 | 18.223 | 9046 | 105421 | 0.75 |
| 36 | 18.476 | 10188 | 130172 | 0.93 |
| 37 | 18.715 | 7809 | 117696 | 0.84 |
| 38 | 19.067 | 4052 | 54395 | 0.39 |
| 39 | 19.312 | 4129 | 51184 | 0.36 |
| 40 | 19.532 | 2298 | 33459 | 0.24 |
| 41 | 19.868 | 2214 | 37053 | 0.26 |
| 42 | 20.667 | 3946 | 29826 | 0.21 |
| 43 | 24.793 | 1778 | 15691 | 0.11 |
| 44 | 26.167 | 1261 | 14346 | 0.10 |
| 45 | 27.886 | 8952 | 112801 | 0.80 |

Peptide Mimocov2 was shown to be very stable upon synthesis.

### Example 3: Vaccination with the Mimocov2 peptide

Test on animal models showed no toxicity.

Test on animals shows that the Mimocov2 vaccine was able to induce an immune response against the RBD detectable with the Siemen test. The immune response due to IgG was still detectable after six months and high enough to provide a protection against SARS-COV-2 infection.

SEQUENCES USED HEREIN

SEQ ID NO: 1 (YP_009724390.1 surface glycoprotein
of the Severe acute respiratory syndrome
coronavirus 2)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
SEQ ID NO: 2 (domain of the S protein responsible
of the interaction with ACE2 receptor)
EGFNCYFPLQSYGFQPTNGVGYQPYR
SEQ ID NO: 3 (Mimocov2 peptide according to the
invention)
EGFNSYFPLQSYGCQPTNGVGYQCYR

1-15. (canceled)

16. A method for modeling a cyclic 3D epitope from a surface of a target antigen comprising the steps of:
a) identifying, in a 3D model representation of the target antigen, one or more conserved region(s) susceptible to be/constitute a 3D epitope, the one or more conserved region(s) possessing one or more charged amino acid(s) selected in the group consisting of arginine (Arg), lysine (Lys), histidine (His), aspartic acid (Asp) and glutamic acid (Glu);
b) performing molecular modeling on the one or more conserved region(s) identified in step a) providing a linear amino acid sequence susceptible to gather the one or more conserved region(s) in the 3D epitope using the following equation to calculate the energy of the force field (equation E):
$$E = \frac{1}{2} {\sum }_{f} {{K}_{f} ( 1 - {e}^{- α ( r - {r}_{0} )} )}^{2} + \frac{1}{2} {\sum }_{θ} {{H}_{θ} ( θ - {θ}_{0} )}^{2} + {\sum }_{Φ} {H}_{Φ} ( 1 + cos {n}_{Φ} ) + {\sum }_{r} \frac{A}{{r}^{12}} - \frac{B}{{r}^{6}} + {\sum }_{r} \frac{q 1 q 2}{D r}$$
wherein E is the energy of the force field, r is the radius between two covalently bound atoms, r₀ is the van der Waals ideal radius at 298 K, θ is the valence angle between two covalent bonds, ϕ is the dihedral angle for SP3 carbons, n is the multiplicity or periodicity of the dihedral angle, the parameters Hϕ, and Hθ are the respective force constants and the variables with the subscript 0 are the respective equilibrium values, A is the distance between two atoms, B is the double distance between two non-bound atoms, q is the atomic charge and D is Debie constant;
c) providing a linear amino acid sequence susceptible to gather the one or more conserved region(s) in the 3D epitope;
d) cyclizing in silico the linear amino acid sequence, so as to mimic the 3D epitope as it is in the target antigen;
e) obtaining a modelized cyclic 3D epitope.

17. The method according to claim 16, wherein the cyclic 3D epitope is a mimotope.

18. The method according to claim 16, wherein the cyclic 3D epitope is synthetic.

19. The method according to claim 16, wherein the target antigen is selected in the group comprising or consisting of a bacterial antigen, a viral antigen and a cancer antigen.

20. The method according to claim 16, wherein step a) is performed by aligning homologous and/or variant sequences of the one or more conserved region(s) to obtain atomic coordinates.

21. The method according to claim 16, wherein the one or more conserved region(s) is/are at the surface of the 3D model representation of the target antigen.

22. The method according to claim 16, wherein step b) comprises performing one or more cycle(s) of energy minimization of a force field induced by atomic coordinates, in particular by the means of one or more energy minimization algorithm(s), wherein the energy minimization is achieved by using the derivate of the equation used in step b).

23. The method according to claim 22, wherein the energy minimization algorithm(s) is/are associated to molecular dynamic steps.

24. The method according to claim 16, wherein step d) is performed chemically, in particular by site specific crosslinking.

25. The method according to claim 16, wherein step d) is performed by substituting 2 amino acid residues by cysteine residues in the linear sequence obtained at step c), and wherein the dihedral angles between each of the alpha-carbons of each of the cysteine residues and any one of the adjacent amino acid residues are compatible with the formation of a disulfide bridge.

26. A cyclic 3D epitope obtained by the synthesis of a cyclic 3D epitope modelized by a method according to claim 16.

27. A method for vaccinating a subject, comprising administering to said subject a prophylactically effective dose of the cyclic 3D epitope according to claim 26.
